(19) United States
(12) Patent Application Publication
(10) Pub. No.: US 2023/0011444 A1
(43) Pub. Date: Jan. 12, 2023

(54) HEMORRHOID BANDING DEVICE AND METHOD FOR BANDING HEMORRHOIDS USING THE SAME

(71) Applicant: Charles Monier, Thibodaux, LA (US)

(72) Inventor: Charles Monier, Thibodaux, LA (US)

(21) Appl. No.: 17/861,369

(22) Filed: Jul. 11, 2022

Related U.S. Application Data
(60) Provisional application No. 63/220,042, filed on Jul. 9, 2021.
(60) Provisional application No. 63/270,712, filed on Oct. 22, 2021.

(51) Int. Cl. A61B 17/12; A61B 17/34

(52) U.S. Cl. CPC A61B 17/12013; A61B 17/3468; A61B 2017/12018; A61B 2017/00367

(57) ABSTRACT

A hemorrhoid banding device for the treatment of hemorrhoidal tissue, the hemorrhoid banding device comprising: a syringe comprising an extended tip, wherein the extended tip has a distal end; a plunger configured to be slidably received in the syringe and movable between (i) a proximal position, and (ii) a distal position; a deployment sheath comprising a distal end and a proximal end, wherein the deployment sheath is configured to slidably fit over the extended tip of the syringe and is configured to move between (i) a proximal position wherein the distal end of the deployment sheath is disposed proximal to the distal end of the extended tip of the syringe, and (ii) a distal position wherein the distal end of the deployment sheath is disposed distal to the distal end of the extended tip of the syringe; a lever comprising a proximal end and a distal end, with the distal end of the lever being hingedly mounted to the syringe; and a link comprising a proximal end and a distal end, wherein the proximal end of the link is hingedly mounted to the plunger and the distal end of the link is hingedly mounted to the proximal end of the lever; wherein the distal end of the link and the proximal end of the lever are displaced further from the syringe when the plunger is in its proximal position than when the plunger is in its distal position.

REFERENCE TO PENDING PRIOR PATENT APPLICATIONS

This patent application claims benefit of:

(1) pending prior U.S. Provisional Patent Application Ser. No. 63/220,042, filed Jul. 9, 2021 by Charles Monier for HEMORRHOID BANDING DEVICE (Attorney's Docket No. MONIER-1 PROV); and

(2) pending prior U.S. Provisional Patent Application Ser. No. 63/270,712, filed Oct. 22, 2021 by Charles Monier for HEMORRHOID BANDING DEVICE (Attorney's Docket No. MONIER-2 PROV).

The two (2) above-identified patent applications are hereby incorporated herein by reference.

### FIELD OF THE INVENTION

This invention relates to surgical apparatus and methods in general, and more particularly to hemorrhoid banding devices and methods for banding hemorrhoids using the same.

### BACKGROUND OF THE INVENTION

The current approach for treating hemorrhoids consists of using a device to apply a small elastic band to the hemorrhoidal tissue to cause necrosis and removal of the excess hemorrhoidal flap. One of the most common banding devices utilizes a syringe to draw in the target tissue using suction. This is accomplished using two hands—one hand to hold the syringe accurately in place in the anal canal while the second hand is utilized to retract the suction plunger of the syringe, thus drawing in the hemorrhoidal tissue. This technique can be affected by hand size, strength and dexterity. This presents an ergonomic problem which can result in inaccurate tissue acquisition and suboptimal treatment. If the wrong location is recognized by the physician, the band must be removed and the banding must be repeated, prolonging the procedure time. If it is not recognized that the band has been placed in the wrong location, the patient will not get relief, and a second appointment will need to be scheduled to repeat the procedure.

Thus there is a need for a hemorrhoid banding device that allows for simple and accurate one-hand operation.

### SUMMARY OF THE INVENTION

The ergonomic challenge referred to above is overcome by the provision and use of a novel hemorrhoid banding device that allows for simple and accurate one-hand operation.

More particularly, in accordance with the present invention, there is provided a hemorrhoid banding device for the treatment of hemorrhoidal tissue, the hemorrhoid banding device comprising:

a syringe comprising an extended tip, wherein the extended tip has a distal end;

a plunger configured to be slidably received in the syringe and movable between (i) a proximal position, and (ii) a distal position;

a deployment sheath comprising a distal end and a proximal end, wherein the deployment sheath is configured to slidably fit over the extended tip of the syringe and is configured to move between (i) a proximal position wherein the distal end of the deployment sheath is disposed proximal to the distal end of the extended tip of the syringe, and (ii) a distal position wherein the distal end of the deployment sheath is disposed distal to the distal end of the extended tip of the syringe;

a lever comprising a proximal end and a distal end, with the distal end of the lever being hingedly mounted to the syringe; and

a link comprising a proximal end and a distal end, wherein the proximal end of the link is hingedly mounted to the plunger and the distal end of the link is hingedly mounted to the proximal end of the lever;

wherein the distal end of the link and the proximal end of the lever are displaced further from the syringe when the plunger is in its proximal position than when the plunger is in its distal position.

In another form of the present invention, there is provided a method for banding hemorrhoidal tissue, the method comprising:

providing a hemorrhoid banding device for the treatment of hemorrhoidal tissue, the hemorrhoid banding device comprising:
a syringe comprising an extended tip, wherein the extended tip has a distal end;
a plunger configured to be slidably received in the syringe and movable between (i) a proximal position, and (ii) a distal position;
a deployment sheath comprising a distal end and a proximal end, wherein the deployment sheath is configured to slidably fit over the extended tip of the syringe and is configured to move between (i) a proximal position wherein the distal end of the deployment sheath is disposed proximal to the distal end of the extended tip of the syringe, and (ii) a distal position wherein the distal end of the deployment sheath is disposed distal to the distal end of the extended tip of the syringe;
a lever comprising a proximal end and a distal end, with the distal end of the lever being hingedly mounted to the syringe; and
a link comprising a proximal end and a distal end, wherein the proximal end of the link is hingedly mounted to the plunger and the distal end of the link is hingedly mounted to the proximal end of the lever;
wherein the distal end of the link and the proximal end of the lever are displaced further from the syringe when the plunger is in its proximal position than when the plunger is in its distal position; and

using the hemorrhoid banding device to apply a band to hemorrhoidal tissue.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a novel hemorrhoid banding device formed in accordance with the present invention, with the hemorrhoid banding device being shown in its pre-deployment condition;

FIG. 2 is a schematic view showing the hemorrhoid banding device of FIG. 1, but with the hemorrhoid banding device shown in its post-deployment condition;

FIG. 3 is a schematic view showing the hemorrhoid banding device of FIG. 1 in a disassembled condition;

FIGS. 4 and 5 are schematic views showing an alternative construction for a portion of the hemorrhoid banding device of FIG. 1;

FIGS. 6-8 are schematic views showing close-ups of selected components of the hemorrhoid banding device of FIG. 1;

FIG. 9 is a schematic view showing a close-up of selected components of the hemorrhoid banding device of FIG. 1;

FIG. 9A is a schematic view showing an alternative construction for the hemorrhoid banding device of FIG. 1;

FIGS. 10-12 and 12A are schematic views showing a split sheath which may be used with a modified form of the hemorrhoid banding device of FIG. 1, with the split sheath being shown in various conditions;

FIGS. 13 and 14 are schematic views showing various ways of operating the hemorrhoid banding device of FIG. 1;

FIGS. 15 and 16 are schematic views showing various features of the hemorrhoid banding device of FIG. 1; and

FIGS. 17-22 are schematic views showing alternative constructions for the hemorrhoid banding device of FIG. 1.

### DETAILED DESCRIPTION OF THE PREFERRED EMBODIMENTS

### The Novel Hemorrhoid Banding Device in General

In one preferred form of the invention, the hemorrhoid banding device of the present invention comprises a linkage mechanism (“link”), a lever, and a thumb slide to controllably locate a target area, apply vacuum to the target area, and deploy a band around the target area by moving a deployment sheath distally along a syringe via the ergonomically positioned thumb slide, all with one-hand operation.

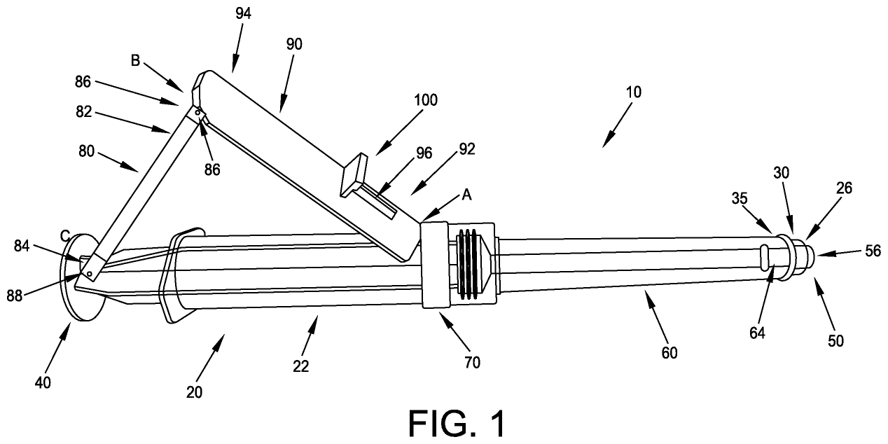

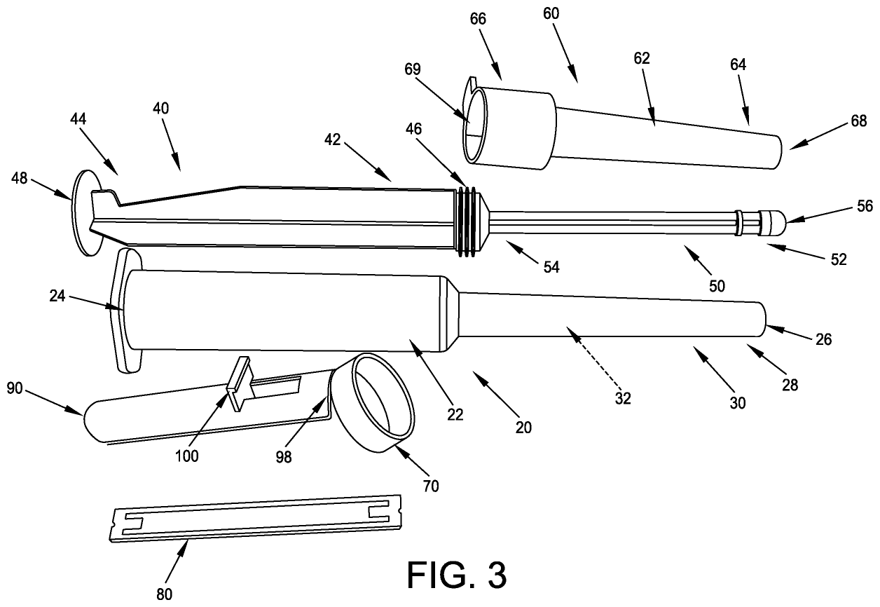

More particularly, and looking now at FIGS. 1-3, there is shown a novel hemorrhoid banding device 10 formed in accordance with the present invention. Hemorrhoid banding device 10 generally comprises a syringe 20 having an extended syringe tip 30 which carries a band 35 (which is a conventional small elastic band of the sort well known in the art of hemorrhoidal banding), a plunger 40 having an obturator 50 at its distal end, a deployment sheath 60 slidably mounted to the extended syringe tip 30 of the syringe 20, a lever ring 70 fixedly mounted to the syringe 20 proximal to the deployment sheath 60, and a link 80 and lever 90 which together hingedly connect the plunger 40 to the lever ring 70. A thumb slide 100 is mounted to the lever 90 in order to allow a user's thumb to move the deployment sheath 60 distally by moving the thumb slide 100 distally.

More particularly, syringe 20 comprises a hollow body 22 having an opening 24 at the proximal end of hollow body 22 and a nozzle 26 at the distal end 28 of extended syringe tip 30. Hollow body 22 is sized to slidably receive plunger 40 via opening 24. Extended syringe tip 30 comprises a cavity 32 which is sized so as to slidably receive obturator 50 and so that a portion of obturator 50 projects out of nozzle 26 of extended syringe tip 30 when hemorrhoid banding device 10 is in its pre-deployment condition (see FIG. 1).

Plunger 40 comprises a distal end 42 and a proximal end 44, with a seal 46 being disposed at its distal end 42 and a plunger flange 48 being disposed at its proximal end 44.

Obturator 50 comprises a distal end 52 and a proximal end 54, with its distal end 52 comprising an atraumatic tip 56 and its proximal end 54 being formed integral with the distal end 44 of plunger 40.

Deployment sheath 60 comprises a hollow body 62 having a distal end 64 and a proximal end 66, with the distal end 64 having a smaller diameter than the proximal end 66. Distal end 64 comprises an opening 68 and proximal end 66 comprises an opening 69. Hollow body 62 is sized so as to slidably receive extended syringe tip 30 of syringe 20 in distal end 64 and a portion of hollow body 22 of syringe 20 in proximal end 66. A portion of extended syringe tip 30 projects out of opening 68 of deployment sheath 60 when hemorrhoid banding device 10 is in its pre-deployment condition.

Link 80 comprises a distal end 82 and a proximal end 84, with distal end 82 being hingedly connected to lever 90 via a pin 86 and with proximal end 84 being hingedly connected to plunger 40 via a pin 88.

Lever 90 comprises a distal end 92, a proximal end 94 and a window 96. Lever ring 70 is hingedly connected to distal end 92 of lever 90 via a living hinge 98. Window 96 is sized so as to slidably receive and guide thumb slide 100.

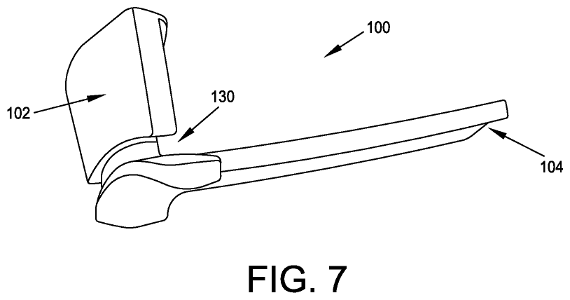

Thumb slide 100 comprises an upright contact surface 102 (FIGS. 7-9) and an element 104 extending distally from the upright contact surface 102. Element 104 is sized so as to slide under lever ring 70 as upright contact surface 102 of thumb slide 100 is advanced distally by a user's thumb. More particularly, as thumb slide 100 is advanced distally by a user's thumb, element 104 slides under lever ring 70 and comes into contact with proximal end 66 of deployment sheath 60 so as to push deployment sheath 60 distally, which in turn pushes band 35 off extended syringe tip 30, as will hereinafter be discussed in further detail.

In its “pre-deployment position” (FIG. 1), the link 80 and lever 90 sit elevated “above” the syringe 20, the distal end 64 of the deployment sheath 60 sits proximal to nozzle 26 of the extended syringe tip 30, the band 35 is mounted to the extended syringe tip 30 just distal to the deployment sheath 60, and the atraumatic tip 56 of the obturator 50 projects out of the nozzle 26 of the extended syringe tip 30.

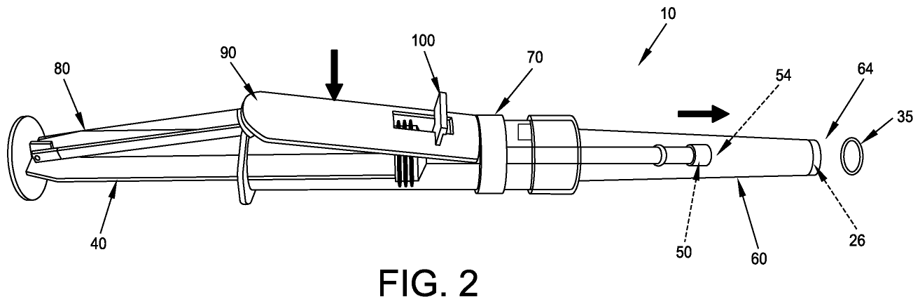

In its “post-deployment position” (FIG. 2), the link 80 and lever 90 sit close to the syringe 20, the distal end 52 of the obturator 50 has been withdrawn into the interior of extended syringe tip 30, the distal end 64 of the deployment sheath 60 sits distal to the nozzle 26 of the extended syringe tip 30, and the band 35 has been deployed off the extended syringe tip 30.

### Use of the Hemorrhoid Banding Device 10

When the hemorrhoid banding device 10 is to be used to band a hemorrhoidal flap, the distal end of the hemorrhoid banding device 10 is inserted into the anus, with the atraumatic tip 56 of the obturator 50 projecting out of the distal end of the hemorrhoid banding device 10 (i.e., the atraumatic tip 56 of the obturator 50 projects out of the nozzle 26 of the extended syringe tip 30). After the distal end of the hemorrhoid banding device 10 has been placed adjacent to the hemorrhoidal flap, the user presses “down” on the lever 90, causing the link 80 and lever 90 to “descend” towards the syringe 20 (FIG. 2). This action causes the plunger 40 to retract, withdrawing the obturator 50 into the interior of the extended syringe tip 30 and applying a vacuum to the extended syringe tip 30, which causes the hemorrhoidal flap to be drawn into the extended syringe tip 30. Once the hemorrhoidal flap is drawn into the extended syringe tip 30, the user pushes the thumb slide 100 distally which causes the deployment sheath 60 to move distally, pushing the band 35 off the extended syringe tip 30 and onto the hemorrhoidal tissue. With the hemorrhoidal flap banded in this manner, the hemorrhoid banding device 10 can be withdrawn from the anus, e.g., by simply pulling hemorrhoid banding device 10 away from the banded hemorrhoidal flap.

### Further Details of the Hemorrhoid Banding Device 10

A. Lever Ring 70, Link 80 and Lever 90

At least one link 80 is utilized to transfer the motion of lever 90 into the linear motion used to retract the syringe plunger 40. The hinge points A, B, C (FIG. 1) can be constructed of pivot pins (e.g., the aforementioned pins 86, 88 as seen at hinge points B and C), or a living hinge (e.g., living hinge 98 as seen at hinge point A). The hinge points B and C can also be constructed using molded pivot pins formed integral with the link 80 or plunger 40, or snap details, or any combination of these constructions.

A preferred embodiment may utilize a molded design employing living hinges at each of the hinge points A, B, C for ease of assembly and cost.

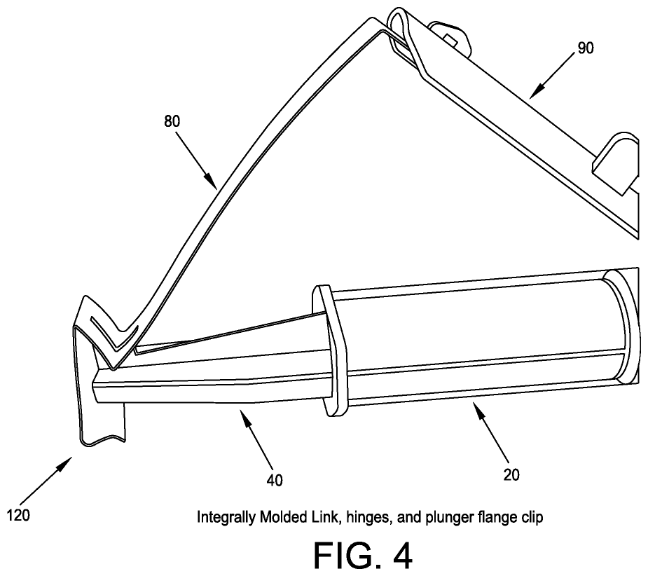

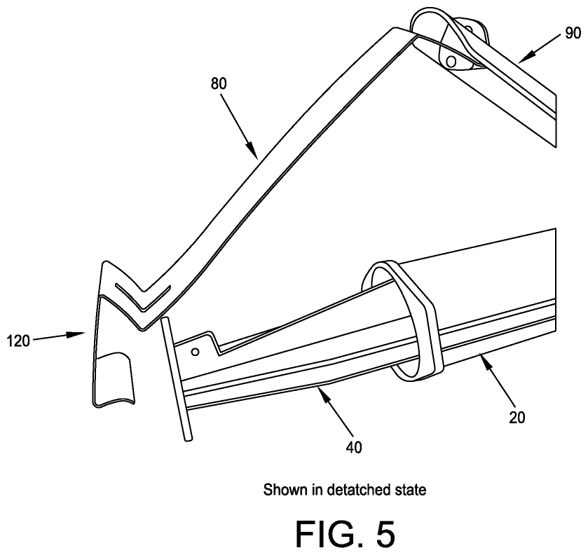

In one form of the invention, and as shown in the embodiment of FIGS. 4 and 5, when the hemorrhoid banding device 10 is formed with a molded design, a plunger flange clip 120 may be used to fixably pivotally attach the link 80 to the plunger 40. In this design, all hinge points may be molded with living hinges to reduce assembly and cost.

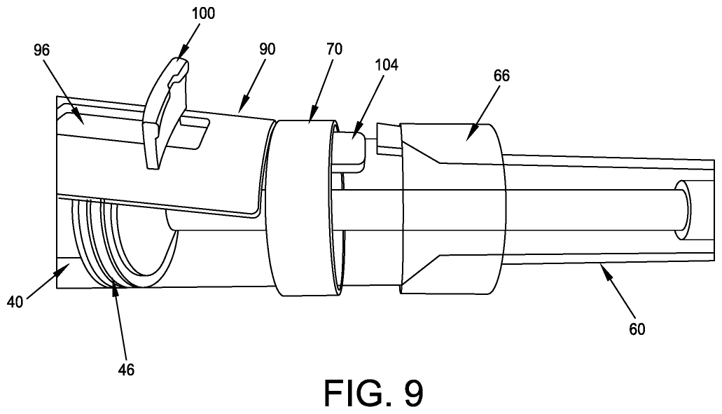

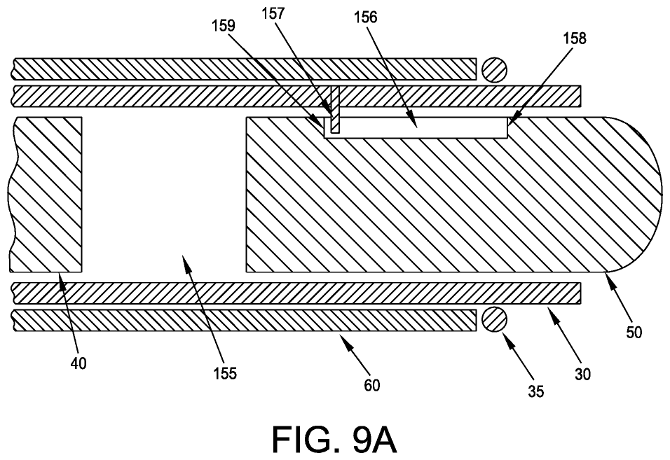

As noted above, the lever 90 may employ a window 96 (see FIG. 9) to guide the thumb slide 100. Lever 90 may also include elements or bosses to ensure that the thumb slide 100 is constrained to linear motion that will not bind. Lubricious materials can be used to reduce sliding friction, or additives can be used in the base material to reduce sliding friction. Additional lubrication in the form of oils, greases, or powders can be applied during assembly as well to provide non-binding motion of the parts.

In another form of the invention, the lever 90 may be directly attached to the hollow body 22 of syringe 20 with a hinge point (instead of being attached to hollow body 22 of syringe 20 via lever ring 70).

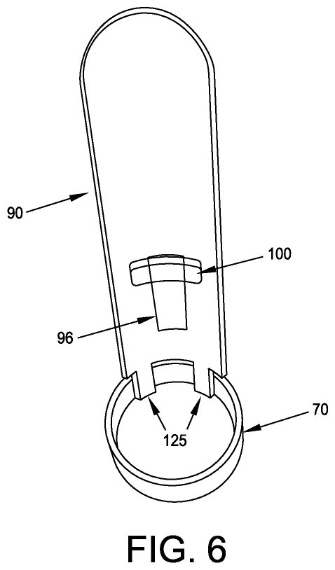

The lever ring 70 may also comprise bosses 125 (FIG. 6) to constrain movement of the thumb slide 100 as the thumb slide 100 travels distally (i.e., the thumb slide 100 is disposed between the bosses 65 so that bosses 65 act as guides for thumb slide 100).

The lever 90 may be molded integrally with the lever ring 70 and link 80 using a polymer with the proper stiffness and living hinge properties to meet the design needs. A polyester is one possible choice for the polymer, with polypropylene being used in a preferred embodiment.

It should be appreciated that the lever ring 70 provides a convenient means to fixably (but pivotally) attach the lever 90 onto the hollow body 22 of the syringe 20.

This may be done with a variety of methods including interference elements molded onto the outer diameter of the syringe 20, whereby the lever ring 70 can be slid over a tapered surface element on syringe 20 that prevents removal of the lever ring 70 (and hence prevents removal of lever 90) once in the desired position.

The lever ring 70 can also be affixed to syringe 20 with adhesives, such as UV acrylic or cyanoacrylates, RF welded, heat-staked, spin-welded or by using other well known polymer attachment methods.

B. Thumb Slide 100

As discussed previously, the thumb slide 100 has an upright contact surface 102 and an element 104 extended distally from this upright contact surface 102 which acts as a deployment tip.

An alternative embodiment can use a living hinge design that is depressed to extend the distal end 64 of the deployment sheath 60 (i.e., in this design, thumb slide 100 can comprise a flexible, concave structure bowed outwardly from, and slidably mounted to, lever 90—depressing the concave structure toward lever 90 causes a portion of the thumb slide 100 to extend distally and drive deployment sheath 60 distally).

The upright contact surface 102 may be enhanced with geometry or surface texture to improve friction with wet gloves. Slots 130 (FIG. 7) may be keyed to the lever window 96 of lever 90.

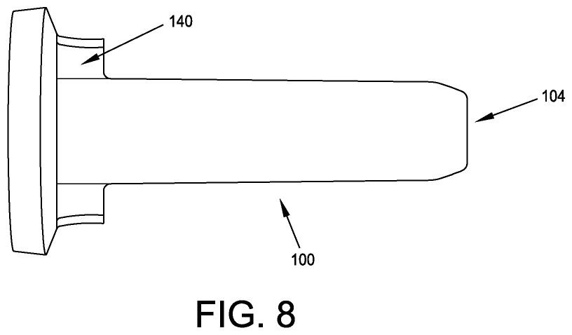

A flanged surface 140 (FIG. 8) parallel with the lever window 96 may be used to stabilize the thumb slide 100 when force is applied in the deployment direction.

It should be appreciated that the material of the thumb slide 100 must be rigid enough to transmit the required force for pushing the deployment sheath 60 distally to deploy the band 35, and may be enhanced by adding ribs or thicker wall sections.

Additionally, the thumb slide 100 may have a degree of flexibility to allow for deflection along the length of the syringe 20 if the lever 90 is not parallel with the syringe 20 at the point of deployment, as shown in FIG. 9.

Another advantage of the thumb slide design is that distal movement of the thumb slide 100 constrains the lever 90 in the depressed position, holding the syringe plunger 40 in the retracted position. This is important in order to maintain the vacuum during band deployment.

Holding the syringe plunger 40 in the retracted position can also be accomplished by employing a detent in the link 80 or any of the hinges A, B, C, or by having the link angle to the syringe 20 be shallow enough to not be overcome by the force of the syringe plunger 40 moving distally in the hollow body 22 of syringe 20.

Note that the link 80 may be a solid cross-section or may employ a geometry which minimizes deflection under load, such as by employing an I-beam construction.

C. Deployment Sheath 60

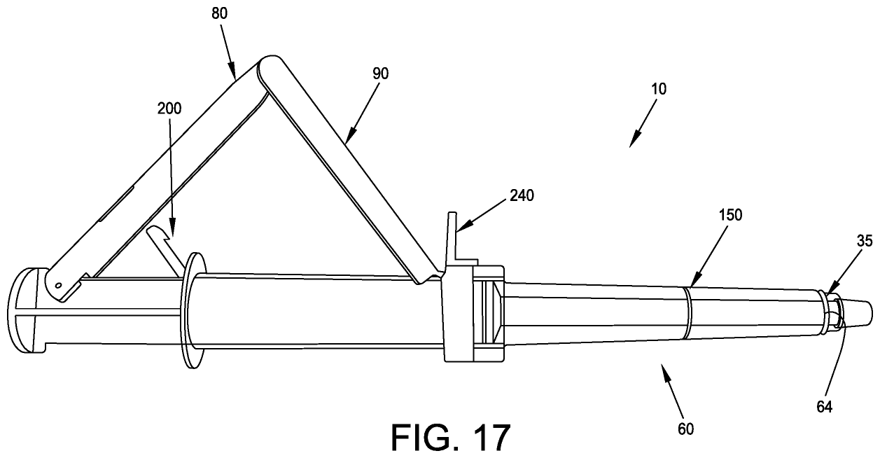

The deployment sheath 60 may have a visible marker 150 (see FIG. 17) approximately 4 cm proximal from its distal end 64, which the physician may use to position at the anal verge. This will locate the distal tip for hemorrhoid band placement proximal to the dentate line. The marker 150 may be a colored ring, added band, produced by paint or ink application, etc.

Releasably attaching the deployment sheath 60 on the extended syringe tip 30 may be accomplished by an interference fit at one or more points in order to retain the deployment sheath 60 on the extended syringe tip 30 during insertion in the rectum prior to deployment of band 35.

D. Obturator 50

In a preferred form of the invention, and as seen in FIGS. 1-3 and 9, obturator 50 is attached to the distal end of plunger 40. To this end, the obturator 50 can be formed integral with the plunger 40, or obturator 50 may be a separate element joined to the plunger 40 during manufacture of the hemorrhoid banding device.

Alternatively, the obturator 50 can be separate from plunger 40. See FIG. 9A. In this form of the invention, an air chamber 155 is provided in the syringe 20 between the distal end of plunger 40 and the proximal end of obturator 50. In this way, when the plunger 40 is inserted distally into the syringe 20 during assembly of the device 10, the air in air chamber 155 initially compresses and then pushes the obturator 50 out of the extended syringe tip 30 of the syringe 20 so as to enable atraumatic insertion into the anus. Once the device 10 is in the desired position in the anus, the plunger 40 is retracted (via link 80 and lever 90), thereby decreasing the pressure in air chamber 155 and creating a vacuum so as to retract the obturator 50 into the extended syringe tip 30 of the syringe 20 which causes tissue to be drawn into the extended syringe tip 30. Once the tissue is drawn into the extended syringe tip 30, the band 35 is pushed off the extended syringe tip 30 onto the tissue by the deployment sheath 60. The hemorrhoid banding device 10 can then by withdrawn from the anus of the patient, e.g., by simply pulling hemorrhoid banding device 10 away from the banded hemorrhoidal flap.

If desired, means can be provided for limiting the distance that obturator 50 can be retracted into extended syringe tip 30 and/or for limiting the distance that obturator 50 can be extended out of extended syringe tip 30. By way of example but not limitation, and looking now at FIG. 9A, a slot 156 can be formed in obturator 50 and a finger 157 can extend from extended syringe tip 30 into slot 156. Engagement of finger 157 with distal end 158 of slot 156 limits the distance that obturator 50 can be retracted into extended syringe tip 30, and engagement of finger 157 with proximal end 159 of slot 156 limits the distance that obturator 50 can be extended out of extended syringe tip 30. Note that limiting the distance that obturator 50 can be retracted into extended syringe tip 30 can be important in limiting the volume of hemorrhoidal tissue captured during banding so as to prevent or decrease the occurrence of inadvertently banding muscle, which is a known cause of acute patient pain. Note also that limiting the distance that obturator 50 can be extended out of extended syringe tip 30 can be important in preventing obturator 50 from accidentally falling out of extended syringe tip 30.

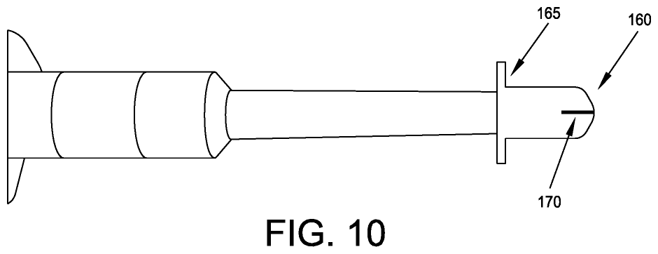

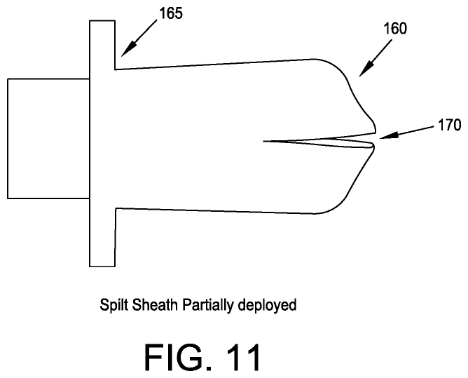

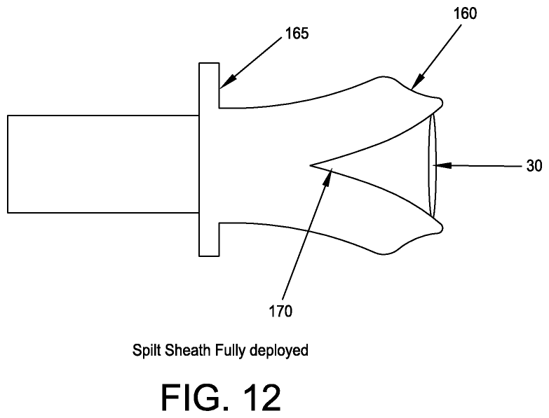

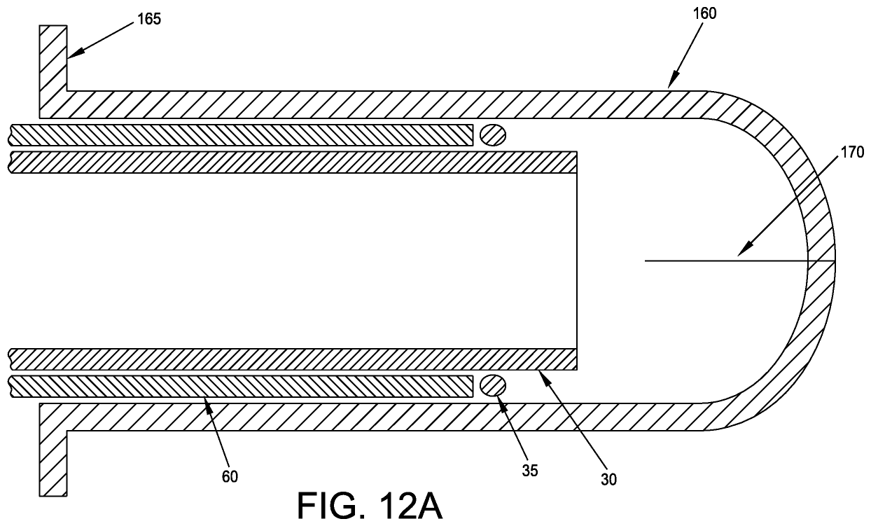

Alternatively, and as shown in FIGS. 10-12 and 12A, the obturator 50 can be eliminated entirely and a split sheath 160 can be used to shield the extended syringe tip 30 from tissue during insertion in the rectum.

The split sheath 160 may be made out of a soft polymer and has a proximal flange 165 and a razor pre-cut 170 allowing the extended syringe tip 30 to protrude out through the split sheath 160 when inserted deeper into the rectum. The proximal flange 165 may be configured so that the physician can manually pull proximally on the proximal flange 165 to expose the extended syringe tip 30, or more preferably, the proximal flange 165 may be configured to function as a stop at the external surface of the anal sphincter, so that continued insertion of the extended syringe tip 30 into the rectum by the physician causes the advancing extended syringe tip 30 to pass through the stopped split sheath 160.

More particularly, the split sheath 160 is configured to be disposed over the distal end 64 of the deployment sheath 60 and the extended syringe tip 30 of the syringe 20 so as to provide an atraumatic surface for entry of the device 10 into the rectum of a patient. As discussed above, in one form of the invention, the proximal flange 165 of the split sheath 160 can be grasped by the surgeon and held in place or retracted as the extended syringe tip 30 is advanced distally. And as discussed above, in another, more preferred, form of the invention, the proximal flange 165 of the split sheath 160 acts as a stop at the external surface of the anal sphincter, preventing further forward movement of the split sheath 160 into the anus. With the proximal flange 165 stopped at the external surface of the anal sphincter, the extended syringe tip 30 carrying the band 35 and the deployment sheath 60 can continue to move forward into the anus, with the razor pre-cut 170 of split sheath 160 being forced open by the advancing extended syringe tip 30 to allow the extended syringe tip 30 so as to protrude out of the split sheath 160. Once the extended syringe tip 30 is in the desired position, the plunger 40 is retracted (via link 80 and lever 90), thereby creating a vacuum at the extended syringe tip 30 of the syringe 20 which causes tissue to be drawn into the extended syringe tip 30. Once the tissue is drawn into the extended syringe tip 30, the deployment sheath 60 is advanced distally to push the band 35 off the extended syringe tip 30 and onto the tissue. The hemorrhoid banding device 10 can then by withdrawn from the anus of the patient, e.g., by simply pulling hemorrhoid banding device 10 away from the banded hemorrhoidal flap.

E. Extended Syringe Tip 30

The outside diameter of the distal end 28 of the extended syringe tip 30 may be produced with a surface texture and/or surface geometry which reduces contact of the band 35, reduces surface friction, and/or lowers the force required for the deployment sheath 60 and thumb slide 100 to deploy the band 35. A frosted texture, and/or an atraumatic spline, are some examples of preferred constructions. Alternatively, a lubricious coating or lubricant may be applied to the extended syringe tip 30 during manufacture or prior to band deployment so as to facilitate deploying band 35 off extended syringe tip 30. Use of low friction additives and materials for the extended syringe tip 30 are other possible solutions to accomplish this goal.

### Additional Notes Regarding The Hemorrhoid Banding Device

The lever, hinges, and link assembly discussed above may be adapted to be retrofit on existing plunger devices or may be provided as a full device with no consideration for retrofitting.

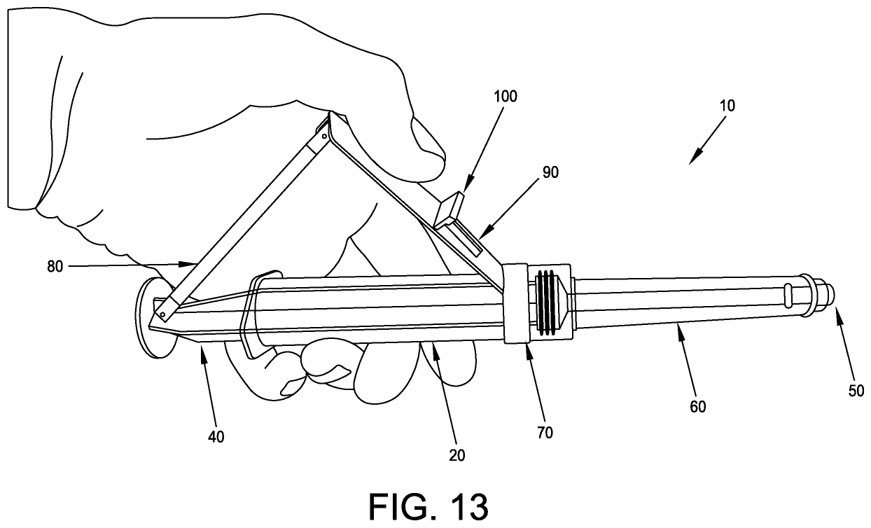

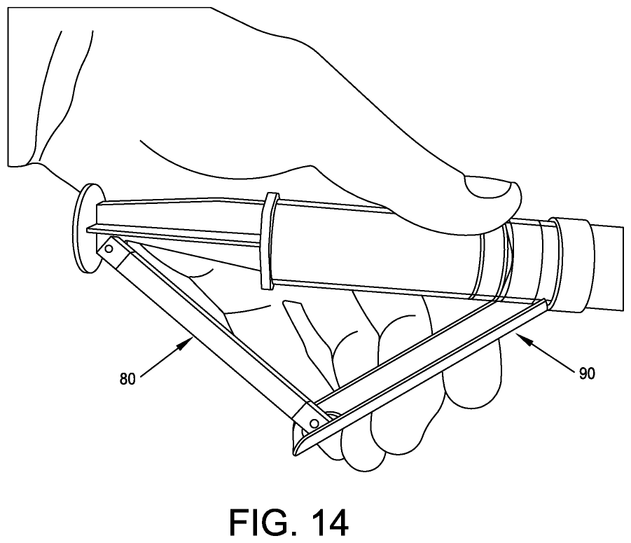

In one preferred form of the invention, the hemorrhoid banding device 10 may be held in the hand of the user so that the lever 90 may be actuated by the user's thumb (FIG. 13); or hemorrhoid banding device 10 may be inverted, with the user's thumb engaging the top of the syringe 20 and the user's fingers actuating the lever 90 (FIG. 14) and with the user's index finger advancing “thumb” slide 100 distally to advance deployment sheath 60 distally.

The utility of this device may also be realized in other applications where single hand operation of a syringe to draw liquid into the syringe is beneficial.

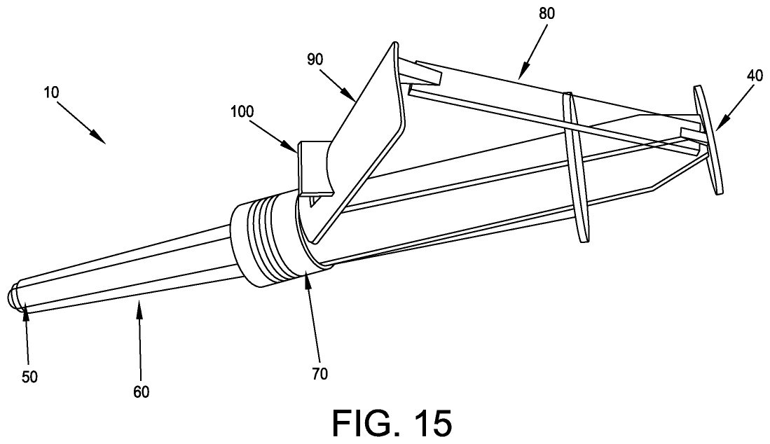

FIG. 15 shows the hemorrhoidal banding device 10 as seen from the proximal end.

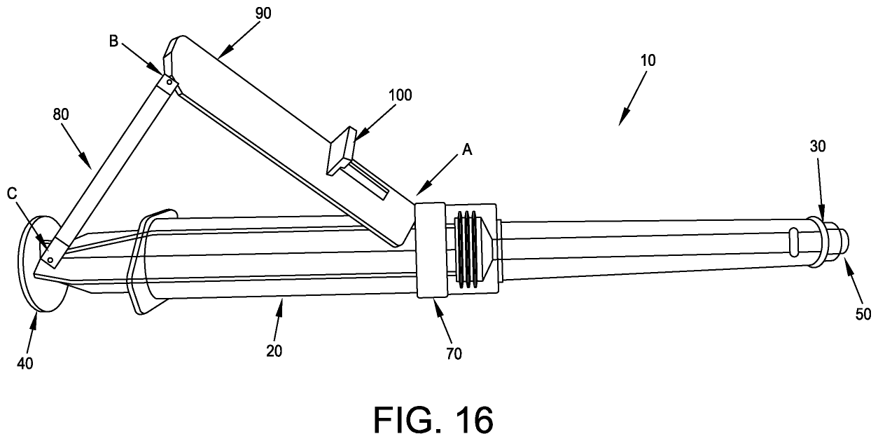

FIG. 16 shows the hemorrhoidal banding device 10 in its “pre-deployment position”, but without the band 35.

### Additional Constructions of the Hemorrhoid Banding Device

A. Integral Locking Tab

As noted above, after the lever 90 has been pushed into its “depressed” condition so that the syringe plunger 40 is in its retracted position, whereby to establish vacuum at the surgical site, the lever 90 may be maintained in its depressed condition by the thumb of the user (or by the fingers of a user if the device 10 is being used in the “inverted position” shown in FIG. 14), or by employing a detent in the link 80 or any of the hinges A, B, C, or by having the link angle to the syringe 20 be shallow enough to not be overcome by the force of the syringe plunger 40 moving distally in the hollow body 22 of syringe 20.

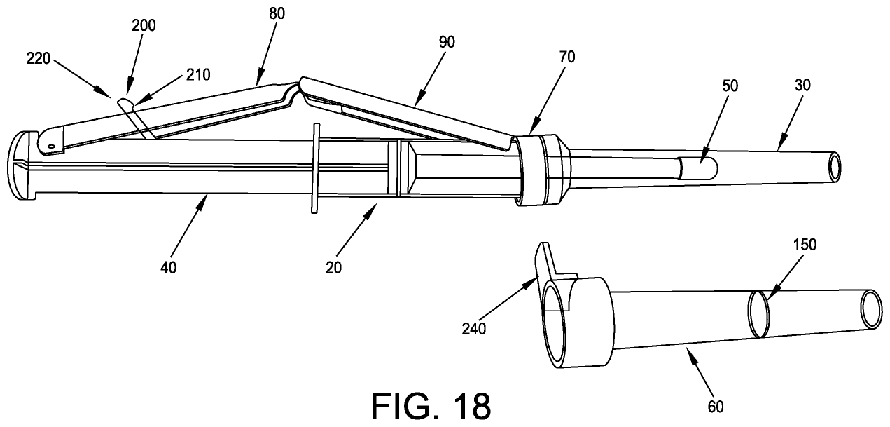

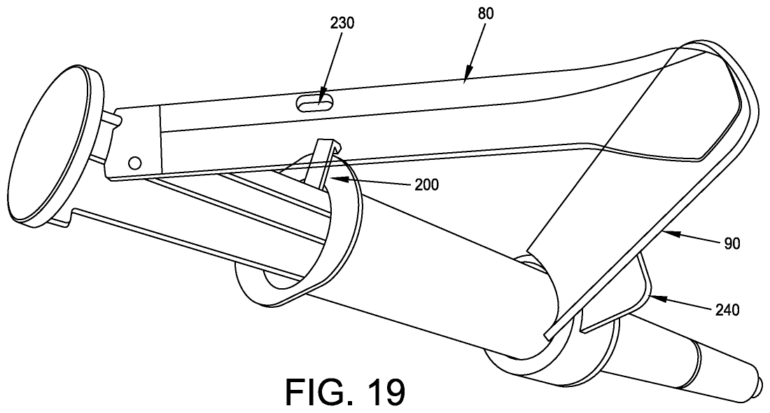

Alternatively, if desired, and looking now at FIGS. 17-19, a locking tab 200 may be provided on the syringe plunger 40 to hold the syringe plunger 40 in its retracted position.

More particularly, the locking tab 200 has a hook 210 on its free end 220, and the link 80 has a slot 230 which receives the locking tab 200. When the lever 90 is depressed and the link 80 drives the syringe plunger 40 rearwardly, the locking tab 200 passes through the slot 230 in the link 80 and the hook 210 catches on the link 80, thereby preventing the link 80 from returning “upwardly” and hence preventing the syringe plunger 40 from moving distally in the hollow body 22 of syringe 20.

B. Deployment Sheath with Thumb Tab

In the construction previously described (e.g., the construction shown in FIGS. 1, 2, 7, 9, etc.), a thumb slide 100 is mounted to the lever 90 and is used to push the deployment sheath 60 distally on the extended syringe tip 30, whereby to push the band 35 off the extended syringe tip 30.

Alternatively, and as seen in FIGS. 17-19, thumb slide 100 may be omitted and a thumb tab 240 may be mounted directly to the deployment sheath 60, with the deployment sheet 60 being moved distally by pressing the thumb tab 240 distally.

C. Link Connection to the Syringe Plunger

In a construction previously described (e.g., the construction shown in FIGS. 1 and 2), the link 80 is shown pivotally connected to the syringe plunger 40 with a pin 88.

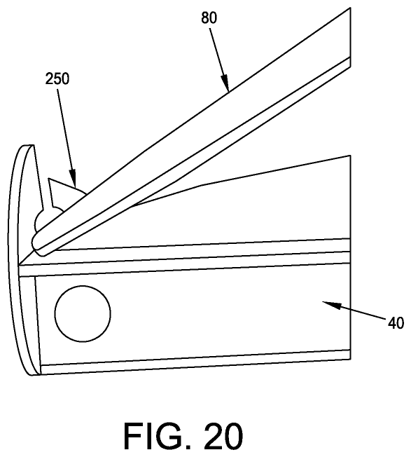

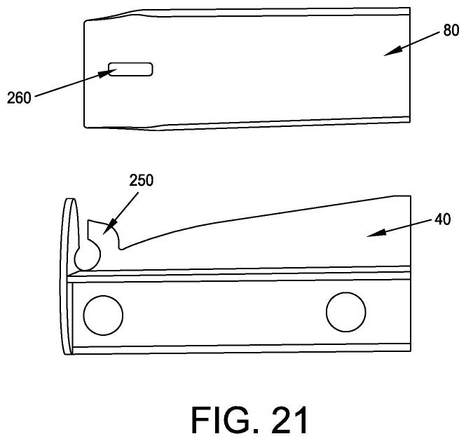

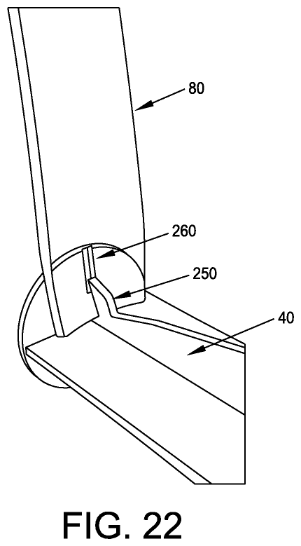

Alternatively, and as shown in FIGS. 20-22, the link 80 can be pivotally connected to the syringe plunger 40 using a finger-and-slot construction.

More particularly, the syringe plunger 40 may comprise a finger 250 at its proximal end 44 just distal to plunger flange 48, and the link 80 may comprise a slot 260 which receives the finger 260, whereby to provide a “pivoting” connection between link 80 and plunger 40.

### Modifications

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A hemorrhoid banding device for the treatment of hemorrhoidal tissue, the hemorrhoid banding device comprising:
a syringe comprising an extended tip, wherein the extended tip has a distal end;
a plunger configured to be slidably received in the syringe and movable between (i) a proximal position, and (ii) a distal position;
a deployment sheath comprising a distal end and a proximal end, wherein the deployment sheath is configured to slidably fit over the extended tip of the syringe and is configured to move between (i) a proximal position wherein the distal end of the deployment sheath is disposed proximal to the distal end of the extended tip of the syringe, and (ii) a distal position wherein the distal end of the deployment sheath is disposed distal to the distal end of the extended tip of the syringe;
a lever comprising a proximal end and a distal end, with the distal end of the lever being hingedly mounted to the syringe; and
a link comprising a proximal end and a distal end, wherein the proximal end of the link is hingedly mounted to the plunger and the distal end of the link is hingedly mounted to the proximal end of the lever;
wherein the distal end of the link and the proximal end of the lever are displaced further from the syringe when the plunger is in its proximal position than when the plunger is in its distal position.

2. A hemorrhoid banding device according to claim 1 wherein the distal end of the lever is pivotally mounted to the syringe by means of a lever ring which is (i) pivotally mounted to the lever and (ii) fixedly mounted to the syringe.

3. A hemorrhoid banding device according to claim 1 further comprising a band disposed on the extended tip of the syringe when the deployment sheath is in its proximal position.

4. A hemorrhoid banding device according to claim 3 wherein a thumb slide is slidably mounted to the lever, wherein the thumb slide comprises a proximal end and a distal end, with the distal end of the thumb slide being disposed adjacent to the proximal end of the deployment sheath, and wherein distal motion of the thumb slide causes distal motion of the deployment sheath, whereby to deploy the band from the extended tip of the syringe.

5. A hemorrhoid banding device according to claim 3 wherein the proximal end of the deployment sheath comprises a thumb tab, whereby distal motion of the thumb tab causes distal motion of the deployment sheath, whereby to deploy the band from the extended tip of the syringe.

6. A hemorrhoid banding device according to claim 1 further comprising an obturator slidably disposed in the syringe distal to the plunger, further wherein the obturator is slidably disposed in the extended tip of the syringe, with a portion of the obturator projecting out of the extended tip of the syringe when the plunger is in its distal position.

7. A hemorrhoid banding device according to claim 6 wherein the obturator is fixed to the plunger.

8. A hemorrhoid banding device according to claim 6 wherein the obturator is separated from the plunger by an air gap.

9. A hemorrhoid banding device according to claim 1 further comprising a split sheath having a proximal flange, wherein the split sheath is configured to be initially disposed over the extended tip of the syringe and the distal end of the deployment sheath.

10. A hemorrhoid banding device according to claim 1 further comprising a locking element to selectively hold the plunger in its proximal position.

11. A method for banding hemorrhoidal tissue, the method comprising:
providing a hemorrhoid banding device for the treatment of hemorrhoidal tissue, the hemorrhoid banding device comprising:
a syringe comprising an extended tip,
wherein the extended tip has a distal end;
a plunger configured to be slidably received in the syringe and movable between (i) a proximal position, and (ii) a distal position;
a deployment sheath comprising a distal end and a proximal end, wherein the deployment sheath is configured to slidably fit over the extended tip of the syringe and is configured to move between (i) a proximal position wherein the distal end of the deployment sheath is disposed proximal to the distal end of the extended tip of the syringe, and (ii) a distal position wherein the distal end of the deployment sheath is disposed distal to the distal end of the extended tip of the syringe;
a lever comprising a proximal end and a distal end, with the distal end of the lever being hingedly mounted to the syringe; and
a link comprising a proximal end and a distal end, wherein the proximal end of the link is hingedly mounted to the plunger and the distal end of the link is hingedly mounted to the proximal end of the lever;
wherein the distal end of the link and the proximal end of the lever are displaced further from the syringe when the plunger is in its proximal position than when the plunger is in its distal position; and
using the hemorrhoid banding device to apply a band to hemorrhoidal tissue.

12. A method according to claim 11 wherein the distal end of the lever is pivotally mounted to the syringe by means of a lever ring which is (i) pivotally mounted to the lever and (ii) fixedly mounted to the syringe.

13. A method according to claim 11 further comprising a band disposed on the extended tip of the syringe when the deployment sheath is in its proximal position.

14. A method according to claim 13 wherein a thumb slide is slidably mounted to the lever, wherein the thumb slide comprises a proximal end and a distal end, with the distal end of the thumb slide being disposed adjacent to the proximal end of the deployment sheath, and wherein distal motion of the thumb slide causes distal motion of the deployment sheath, whereby to deploy the band from the extended tip of the syringe.

15. A method according to claim 13 wherein the proximal end of the deployment sheath comprises a thumb tab, whereby distal motion of the thumb tab causes distal motion of the deployment sheath, whereby to deploy the band from the extended tip of the syringe.

16. A method according to claim 11 further comprising an obturator slidably disposed in the syringe distal to the plunger, further wherein the obturator is slidably disposed in the extended tip of the syringe, with a portion of the obturator projecting out of the extended tip of the syringe when the plunger is in its distal position.

17. A method according to claim 16 wherein the obturator is fixed to the plunger.

18. A method according to claim 16 wherein the obturator is separated from the plunger by an air gap.

19. A method according to claim 11 further comprising a split sheath having a proximal flange, wherein the split sheath is configured to be initially disposed over the extended tip of the syringe and the distal end of the deployment sheath.

20. A method according to claim 11 further comprising a locking element to selectively hold the plunger in its proximal position.
